(19) United States
(12) Patent Application Publication
(10) Pub. No.: US 2025/0311790 A1
(43) Pub. Date: Oct. 9, 2025

(54) REUSABLE CLOTHING ARTICLE AND METHOD OF ITS MANUFACTURE

(71) Applicant: TEFRON LTD., Misgav (IL)

(72) Inventors: Laurie Christina POLLICINO, Massapequa, NY (US); Marianne VILLENEUVE, Mirabel (CA)

(21) Appl. No.: 18/865,734

(22) PCT Filed: May 15, 2023
(86) PCT No.: PCT/IL2023/050498

§ 371 Date: Nov. 14, 2024

Related U.S. Application Data
(60) Provisional application No. 63/364,744, filed on May 16, 2022.

(51) Int. Cl. A41B 9/12; A61F 13/15; A61F 13/496; A61F 13/539; D04B 1/10

(52) U.S. Cl. CPC A41B 9/12; A61F 13/15268; A61F 13/496; A61F 13/539; D04B 1/102; A61F 2013/15276; D10B 2509/026

(57) ABSTRACT

A layer for a reusable liquid-absorbent article of clothing, configured to constitute an upper layer of an absorbent pad intended for contact with a body of a user, is formed of a knitted fabric. The layer comprises a pattern of areas knitted so as to have different liquid permeabilities in a through-thickness direction of the layer, including areas of high permeability and areas of restricted permeability, the areas being mutually arranged so as to inhibit movement of liquid towards the side edges at least along a portion of their length. A reference plane passes between the top and bottom faces, wherein at least a majority of the areas of high permeability extend from the reference plane towards the bottom face, and at least a majority of the areas of restricted permeability extend from the reference plane towards the top face.

### TECHNOLOGICAL FIELD

The present disclosure relates to an absorbent clothing article comprising a pad, such as but not limited to incontinence, post-partum or period underwear, and in particular to a moisture wicking upper layer of the pad fabricated from a circular knit fabric.

### BACKGROUND

References considered to be relevant as background to the presently disclosed subject matter are listed below:
U.S. Pat. No. 6,927,182, issued Aug. 9, 2005 to Malden Mills Industries, Inc. entitled Enhanced Composited Sweatshirt Fabric with Knit Constructed Channels.
EP 3895675 published Oct. 20, 2021 to Kajanthan et al, entitled Absorbent Pad for a Garment.
U.S. Pat. No. 5,906,876 issued May 25, 1999, to David Conway, entitled Absorbent Fabric and Undergarments Incorporating the Fabric.
WO 2011/108954 published Sep. 9, 2011 to Esteves De Sousa Fangueiro et al, entitled An Integrated, Washable and Reusable Three-Dimensional (3D) Multifunctional Knitted Fabric Structure and Method to Produce the Same.
US 2021/0290447 published Sep. 23, 2021 to Sepello et al, entitled Durable Absorbent Pant

Acknowledgement of the above references herein is not to be inferred as meaning that these are in any way relevant to the patentability of the presently disclosed subject matter.

### GENERAL DESCRIPTION

Absorbent underwear is reusable underwear that is similar to conventional underwear, and can be found in various styles, e.g. boy shorts, bikinis, briefs and thongs. The key difference is that while absorbent underwear might look like standard underwear, it has a sewn-in or easily replaceable absorbent pad that pulls moisture away from the user-contacting upper surface. Ideally, the absorbent pad is very thin with no bulging, adhesive, wings, or any of the other inconveniences such as significant environmental waste contribution that come with traditional disposable pads. The absorbent underwear works to absorb blood, urine and other discharge fluids, and like conventional pads and pantyliners on the market, there can be varying levels of absorption, e.g. being designed for light, moderate or heavy flows. Conventional absorbent underwear with permanently sewn-in pads is formed by first creating a pad comprising multiple layers cut out of fabric, including an upper, moisture-wicking layer, at least one absorbent middle layer and a lower fluid-impervious layer. The upper layer can be cut from a fabric which can have a repeating textured pattern designed to enhance wicking of moisture and fluid away from one surface of the upper layer, through the fabric towards the opposite surface. These layers are then stitched, bonded or otherwise joined to a gusset-portion of a pair of underwear.

In some embodiments the absorbent underwear should have maximal absorption capabilities without leakage, in order to prevent the need to change the underwear so frequently, and which would avoid embarrassing situations for the wearer in a public setting. It would also reduce the need to purchase and launder so many pairs of absorbent underwear and thus reduce the environmental impact of using absorbent underwear.

In some embodiment, the upper layer of the pad can provide not only a wicking function but also a fluid directing function, both of which can be adjusted to vary across the upper layer, for example so as to provide different wicking or fluid-transfer rates at different locations, as well as facilitating movement of fluid towards predetermined areas for subsequent wicking there. This realization facilitates fluid wicking and subsequent absorption in the middle layer(s) of the pad at a greater spread of locations across the pad, and enables faster wicking in various locations other than where the fluid is applied to the pad upper layer. In this way, fluid absorption capabilities of the pad can be increased by making use of more absorbent area of the pad, while also inhibiting movement of fluid towards edges of the pad from which it would be otherwise more likely to leak at a lower overall absorption level of the pad. Additionally, by spreading the fluid more evenly across the pad, thinner, more streamlined pads can be produced, additionally reducing visibility of the existence of the pad, such as when wearing absorbent swimwear underwear (e.g. bikini style) and thus further reducing embarrassment to a wearer in social settings.

The present disclosure provides a layer for a reusable liquid-absorbent article configured to constitute an upper layer of a pad intended for contact with a body of a user, the layer being formed of a knitted fabric, and comprising:
a top face and an opposite bottom face defining a through-thickness direction of the layer therebetween;
a front edge, a rear edge and two side edges extending therebetween, a longitudinal direction being a direction spanning between the front and rear edges, and a transverse direction being a direction spanning between the two side edges;
a longitudinal central axis extending in the longitudinal direction between the front and rear edges so as to bisect the layer evenly;
a transverse central axis extending between the two side edges and crossing the longitudinal central axis at a central intersection point of the layer;
a pattern of knitted areas configured to have different degrees of liquid permeability in the through-thickness direction of the layer, including areas of high permeability and areas of restricted permeability, mutually arranged so as to inhibit movement of liquid towards the side edges at least along a portion of their length, each area having a height along the through-thickness direction, each area having a height along the through thickness direction, each area having a height along the through-thickness direction;
a reference plane passing between the top and bottom faces and parallel to the longitudinal and transverse axes, at least a majority of the areas of high permeability being disposed with most of their height extending downwardly from the reference plane, and at least a majority of the areas of restricted permeability being disposed with most of their height extending upwardly from the reference plane;
wherein the layer is cut from a tubular fabric piece formed by a knitting process, and the pattern of areas is formed by selectively manipulating and arranging threads of the tubular fabric piece relative to each other during the knitting process.

The pattern of knitted areas provide different zones of permeability, in which areas of restricted permeability direct fluids to the areas of high permeability, so that the fluids can be wicked to the absorbent pad below.

In any of the embodiments the different degrees of permeability can be defined by different degrees of porosity, which for a predetermined yarn thickness is determined by a combination including knitting density and profile of the layer in a cross-section thereof taken perpendicular to the reference plane.

In any of the embodiments the reusable liquid-absorbent article can comprise a crotch pad.

In any of the embodiments the pattern of knitted areas can comprise:
at least one area of relatively high permeability, configured to facilitate passage of liquid in the through-thickness direction of the layer, the at least one area of relatively high permeability extending in the longitudinal direction and being spaced from the two side edges and located centrally in the transverse direction; and
at least two areas of restricted permeability extending so as to bound the at least one area of relatively high permeability at least partially along longitudinal sides thereof, the at least two areas of restricted permeability being knitted in a different arrangement from that of the at least one area of relatively high permeability, so as to inhibit passage of a liquid laterally from the at least one area of relatively high permeability towards the two side edges.

The areas of restricted permeability direct the flow of fluids away from the sides of the article to the areas of high permeability to be passed to the absorbent material below.

In any of the embodiments, the at least one area of relatively high permeability has a height extending from above the reference plane downwardly below the reference plane, and comprises a first textured surface having protrusions extending above the reference plane, and recesses extending downwardly below the reference plane, optionally wherein the recesses comprise recessed channels, further optionally wherein the recessed channels circumscribe the protrusions.

In any of the embodiments, the at least one area of relatively high permeability can have a tessellated texture, optionally comprising a grid or honeycomb arrangement of the protrusions and the recesses. The honeycomb arrangement provides a highly permeable area through which the liquids can pass.

In any of the embodiments the at least one area of relatively high permeability can comprise a plurality of openings formed in the protrusions other than regular spacing between the threads, the openings passing through the layer in the through-thickness direction, configured for passage of liquid therethrough.

In any of the embodiments, the at least two areas of restricted permeability can have a height extending from below the reference plane towards the top face.

The raised areas of restricted permeability form a wall against the passage of liquids out of the areas of high permeability.

In any of the embodiments, the at least two areas of restricted permeability can be joined together at longitudinal ends thereof by front and rear joining portions to form a loop-shape.

In any of the embodiments, at least one of the front and rear joining portions of the loop-shape can be knitted in a similar manner to the at least two areas of restricted permeability and extends in a discontinuous manner, so as to provide an area of low permeability, configured to restrictively allow passage of a liquid through the layer at least in a longitudinal direction from one side of the respective joining portion to another side.

At the front and/or rear ends of the different areas, ports can be formed by alternating between high/medium permeability areas and restricted permeability areas enabling fluids to pass into the areas of high permeability.

In any of the embodiments, at least one area of intermediate permeability can be knitted in a different arrangement from the at least one area of relatively high permeability and the at least two areas of restricted permeability so as to allow lateral movement of a liquid in a direction having a component in the longitudinal direction, and can have a height extending from above the reference plane towards the bottom face to a lesser extent than the at least one area of relatively high permeability, the at least one area of intermediate permeability can be at least partially bounded by at least one area of restricted permeability.

In any of the embodiments the at least one area of intermediate permeability can comprise a second textured surface having alternately arranged ridges and channels, the ridges protruding upwardly above the reference plane and the channels being recessed downwardly towards the bottom face; optionally wherein the ridges and channels can be arranged extending at an angle between the longitudinal and transverse directions.

In any of the embodiments, the at least one area of intermediate permeability can have a twilled rib texture.

The twilled rib texture provides alternating ridges and channels enabling both passage of fluid therethrough and direction of fluid to the highly absorbent areas.

In any of the embodiments the area of restricted permeability can have a raised line texture.

In any of the embodiments the threads can comprise at least one of graphene, nylon or spandex fibers.

In any of the embodiments the layer can comprise a chemically applied wicking finish.

In any of the embodiments the knitting process can be a circular knitting process.

The circular knitting process enables a single layered jersey to be fabricated in a single knitting run.

The present disclosure also provides a tubular fabric piece comprising the layer according to any one of above embodiments formed integrally thereon;
wherein the tubular fabric piece comprises cut guidelines along which the layer can be cut, the cut guidelines demarcating the front edge, the rear edge and the two side edges.

The present disclosure also provides a reusable liquid-absorbent crotch pad comprising: an upper layer comprising a layer as claimed in any one of above embodiments; at least one intermediate absorbent layer; and a liquid-impermeable lower layer; wherein the upper layer, the at least one intermediate absorbent layer and the liquid-impermeable lower layer are secured together along peripheral edges thereof.

The present disclosure also provides a reusable panty comprising the reusable liquid-absorbent crotch pad according to any of the above embodiments, optionally wherein the reusable liquid-absorbent crotch pad can be attached to a gusset of the panty along only some of the peripheral edges of the reusable liquid-absorbent crotch pad.

The present disclosure also provides a method of producing a layer for a reusable liquid-absorbent article according to any one of above embodiments, comprising:
knitting a tubular fabric piece comprising the pattern of areas surrounded by cut guidelines along which the layer can be cut from the tubular fabric piece, the cut guidelines demarcating the front edge, the rear edge and the two side edges of the layer;
wherein knitting comprises a circular weft knitting process; and
subsequently cutting out the layer from the tubular fabric piece along the cut guidelines.

The present disclosure also provides an upper wicking layer for reusable absorbent underwear configured for contact with a body of a user, the upper wicking layer being cut from a circular knitted fabric and comprising:
side edges, a front and a rear,
a first zone of higher permeability at least partially bounded by a second zone of lesser permeability on one side thereof and a third zone of lesser permeability on another side thereof, the second zone of lesser permeability and the third zone of lesser permeability extending outwardly from either side of the first zone configured to inhibit movement of liquid applied to the first zone towards the side edges;
wherein the first zone of higher permeability has:
a first degree of porosity of the knitted fabric;
wherein the second and third zones of lesser permeability has:
a second degree of porosity of the knitted fabric, the first degree of porosity is greater than the second degree of porosity.

It is noted that the layer for a reusable liquid-absorbent article can be designed in any suitable manner such as with alterative arrangements, contours, shapes, sizes and the like.

### EMBODIMENTS

A more specific description is provided in the Detailed Description whilst the following are non-limiting examples of different embodiments of the presently disclosed subject matter.
1. A layer for a reusable liquid-absorbent article configured to constitute an upper layer of a pad intended for contact with a body of a user, the layer being formed of a knitted fabric, and comprising:
   a top face and an opposite bottom face defining a through-thickness direction of the layer therebetween;
   a front edge, a rear edge and two side edges extending therebetween, a longitudinal direction being a direction spanning between the front edge and the rear edge, and a transverse direction being a direction spanning between the two side edges;
   a longitudinal central axis extending in the longitudinal direction between the front and rear edges so as to bisect the layer evenly;
   a transverse central axis extending between the two side edges and crossing the longitudinal central axis at a central intersection point of the layer;
   a pattern of knitted areas configured to have different degrees of liquid permeability in the through-thickness direction of the layer, including areas of high permeability and areas of restricted permeability, mutually arranged so as to inhibit movement of liquid towards the side edges at least along a portion of their length, each area having a height along the through-thickness direction; and
a reference plane passing between the top and bottom faces and parallel to the longitudinal central axis and the transverse central axis, at least a majority of the areas of high permeability being disposed with most of their height extending downwardly from the reference plane, and at least a majority of the areas of restricted permeability being disposed with most of their height extending upwardly from the reference plane,
wherein the layer is cut from a tubular fabric piece formed by a knitting process, and the pattern of knitted areas is formed by selectively manipulating and arranging threads of the tubular fabric piece relative to each other during the knitting process.
2. The layer according to Embodiment 1, wherein the different degrees of liquid permeability is defined by different degrees of porosity, which for a predetermined yarn thickness is determined by a combination including: knitting density and profile of the layer in a cross-section thereof taken perpendicular to the reference plane.
3. The layer according to Embodiment 1 or 2, wherein the reusable liquid-absorbent article comprises a crotch pad.
4. The layer according to any one of Embodiments 1 to 3, wherein the pattern of knitted areas comprises:
   at least one area of relatively high permeability, configured to facilitate passage of liquid in the through-thickness direction of the layer, the at least one area of relatively high permeability extending in the longitudinal direction and being spaced from the two side edges and located centrally in the transverse direction; and
   at least two areas of restricted permeability extending so as to bound the at least one area of relatively high permeability at least partially along longitudinal sides thereof, the at least two areas of restricted permeability being knitted in a different arrangement from that of the at least one area of relatively high permeability, so as to inhibit passage of a liquid laterally from the at least one area of relatively high permeability towards the two side edges.
5. The layer according to Embodiment 4, wherein the at least one area of relatively high permeability has a height extending from above the reference plane downwardly below the reference plane, and comprises a first textured surface having protrusions extending above the reference plane, and recesses extending downwardly below the reference plane, optionally wherein the recesses comprise recessed channels, further optionally wherein the recessed channels circumscribe the protrusions.
6. The layer according to Embodiment 5, wherein the at least one area of relatively high permeability has a tessellated texture, optionally comprising a grid or honeycomb arrangement of the protrusions and the recesses.
7. The layer according to Embodiment 5 or 6, wherein the at least one area of relatively high permeability comprises a plurality of openings formed in the protrusions other than regular spacing between the threads, the openings passing through the layer in the through-thickness direction, configured for passage of liquid therethrough.
8. The layer according to any one of Embodiments 4 to 7, wherein the at least two areas of restricted permeability have a height extending from below the reference plane towards the top face.
9. The layer according to any one of Embodiments 4 to 8, wherein the at least two areas of restricted permeability are joined together at longitudinal ends thereof by front and rear joining portions to form a loop-shape.
10. The layer according to Embodiment 9 wherein at least one of the front and rear joining portions of the loop-shape is knitted in a similar manner to the at least two areas of restricted permeability and extends in a discontinuous manner, so as to provide an area of low permeability, configured to restrictively allow passage of liquid through the layer at least in the longitudinal direction from one side of the respective joining portion to another side.
11. The layer according to any one of Embodiments 4 to 10, further comprising at least one area of intermediate permeability which is knitted in a different arrangement from the at least one area of relatively high permeability and the at least two areas of restricted permeability, so as to allow lateral movement of liquid in a direction having a component in the longitudinal direction, and has a height extending from above the reference plane towards the bottom face to a lesser extent than the at least one area of relatively high permeability, the at least one area of intermediate permeability being at least partially bounded by at least one area of restricted permeability.
12. The layer according to Embodiment 11, wherein the at least one area of intermediate permeability comprises a second textured surface having alternately arranged ridges and channels, the ridges protruding upwardly above the reference plane and the channels being recessed downwardly towards the bottom face, optionally wherein the ridges and channels are arranged extending at an angle between the longitudinal and transverse directions.
13. The layer according to Embodiment 11 or 12, wherein the at least one area of intermediate permeability has a twilled rib texture.
14. The layer according to any one of Embodiments 1 to 13, wherein the area of restricted permeability has a raised line texture.
15. The layer according to any one of Embodiments 1 to 14, wherein the threads comprise at least one of graphene, nylon or spandex fibres.
16. The layer according to any one of Embodiments 1 to 15, comprising a chemically applied wicking finish.
17. The layer according to any one of Embodiments 1 to 16, wherein the knitting process is a circular knitting process.
18. A tubular fabric piece comprising the layer according to any one of Embodiments 1 to 17 formed integrally thereon;
   wherein the tubular fabric piece comprises cut guidelines along which the layer can be cut, the cut guidelines demarcating the front edge, the rear edge and the two side edges.
19. A reusable liquid-absorbent crotch pad comprising:
   an upper layer comprising a layer as described in any one of Embodiments 1 to 17;
   at least one intermediate absorbent layer; and
   a liquid-impermeable lower layer;
   wherein the upper layer, the at least one intermediate absorbent layer and the liquid-impermeable lower layer are secured together along peripheral edges thereof.
20. A reusable panty comprising the reusable liquid-absorbent crotch pad according to Embodiment 19, optionally wherein the reusable liquid-absorbent crotch pad is attached to a gusset of the panty along only some of the peripheral edges of the reusable liquid-absorbent crotch pad.
21. A method of producing a layer for a reusable liquid-absorbent article according to any one of Embodiments 1 to 17, comprising:
   knitting a tubular fabric piece comprising the pattern of areas surrounded by cut guidelines along which the layer can be cut from the tubular fabric piece, the cut guidelines demarcating the front edge, the rear edge and the two side edges of the layer;
   wherein knitting comprises a circular weft knitting process; and
   subsequently cutting out the layer from the tubular fabric piece along the cut guidelines.
22. An upper wicking layer for reusable absorbent underwear configured for contact with a body of a user, the upper wicking layer being cut from a circular knitted fabric and comprising:
   side edges, a front and a rear,
   a first zone of higher permeability at least partially bounded by a second zone of lesser permeability on one side thereof and a third zone of lesser permeability on another side thereof, the second zone of lesser permeability and the third zone of lesser permeability extending outwardly from either side of the first zone configured to inhibit movement of liquid applied to the first zone towards the side edges;
   wherein the first zone of higher permeability has:
      a first degree of porosity of the knitted fabric;
   wherein the second and third zones of lesser permeability have:
      a second degree of porosity of the knitted fabric, the first degree of porosity is greater than the second degree of porosity.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates an exemplary pair of absorbent underwear in accordance with the present disclosure;

FIG. 2 illustrates an example of an upper wicking layer of the pair of absorbent underwear of FIG. 1;

FIG. 3 illustrates another example of an upper wicking layer of the pair of absorbent underwear of FIG. 1;

FIG. 4 illustrates a portion of the upper wicking layer of FIG. 3;

FIG. 5 illustrates a cross-section of the upper wicking layer of FIG. 2 taken along a longitudinal central axis L-L shown in FIG. 3;

FIG. 6A illustrates a portion of a texture of an exemplary area of high permeability of the upper wicking layer of FIG. 3;

FIG. 6B illustrates an enlarged cross-sectional profile of the texture shown in FIG. 6A;

FIG. 7A illustrates a portion of a texture of an exemplary area of intermediate permeability of the upper wicking layer of FIG. 3;

FIG. 7B illustrates an enlarged cross-sectional profile of the texture shown in FIG. 7A;

FIG. 8A illustrates an enlarge cross-sectional profile of a texture of an exemplary area of restricted permeability;

FIG. 8B illustrate an enlarged cross-section profile of the textures shown in FIG. 8A; and

FIG. 9 illustrates an enlarged section of an exemplary area of high permeability of the upper wicking layer of FIG. 2 and FIG. 3.

### DETAILED DESCRIPTION

The following detailed description sets forth general and specific details about features of the adaptors according to various aspects and examples of the presently disclosed subject matter.

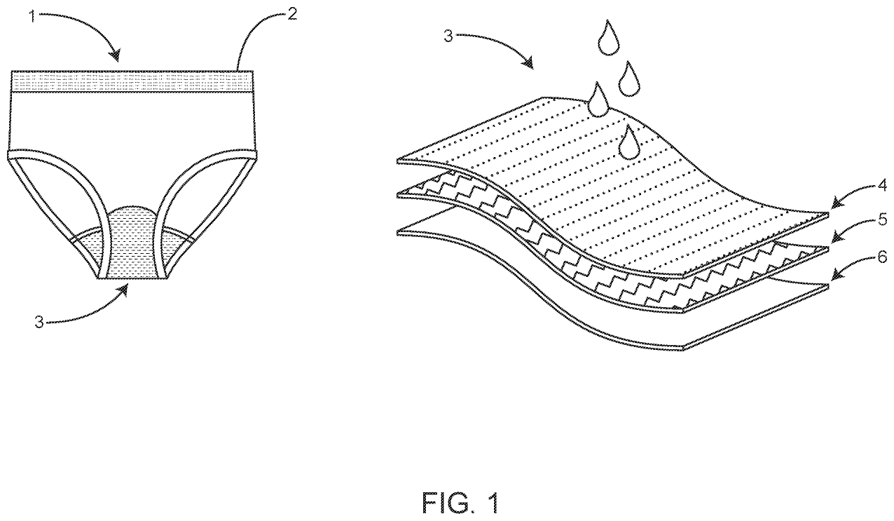

With reference to FIG. 1, a pair of absorbent underwear 1 includes a pair of underwear 2 having an absorbent pad 3 attached to the gusset. The absorbent pad 3 includes an upper wicking layer 4, at least one middle or intermediate absorbent layer 5, and a lower fluid-impermeable layer 6. The at least one middle absorbent layer 5 can be comprised of any suitable washable super-absorbent thin material, e.g. superabsorbent polymer (SAP), and polyester, or any other material suitable for absorbing liquids or gases such as bodily fluids. The lower fluid-impermeable layer 6 can be comprised of any suitable fluid impervious material, e.g. thermoplastic or thermoset film, configured to prevent the flow of any fluid, which may comprise a liquid or a gas.

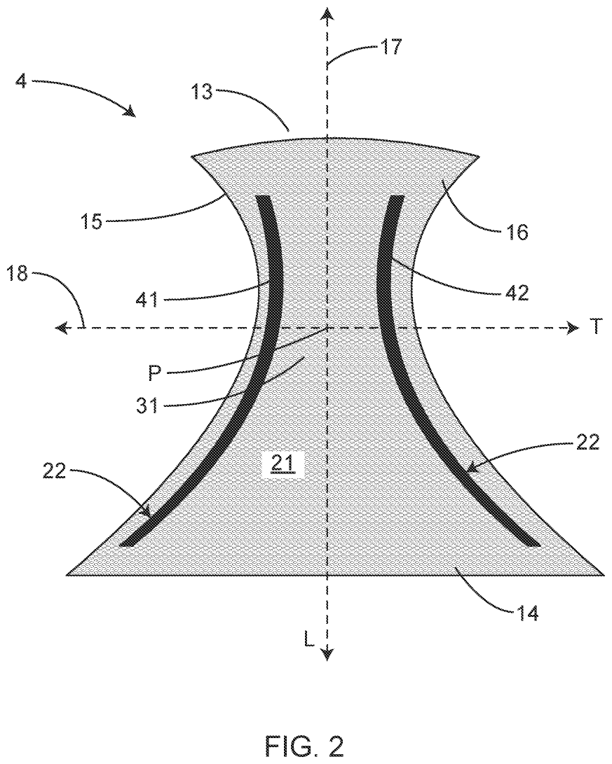

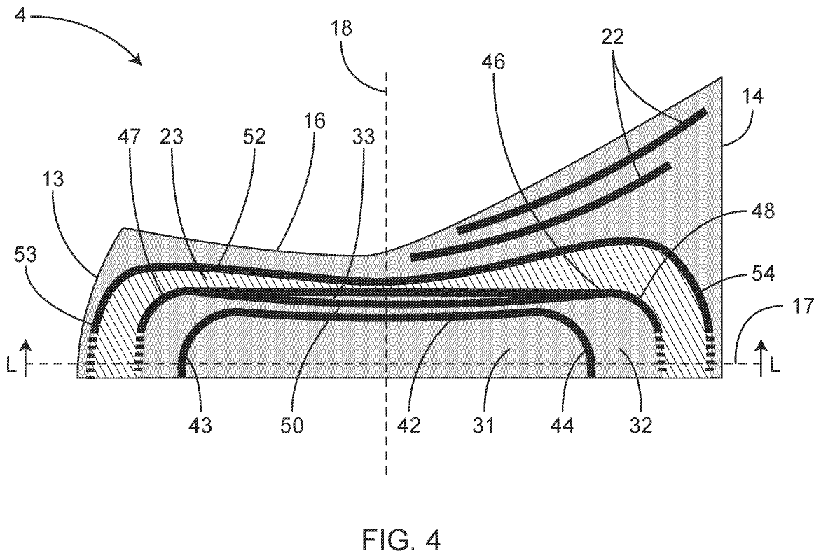

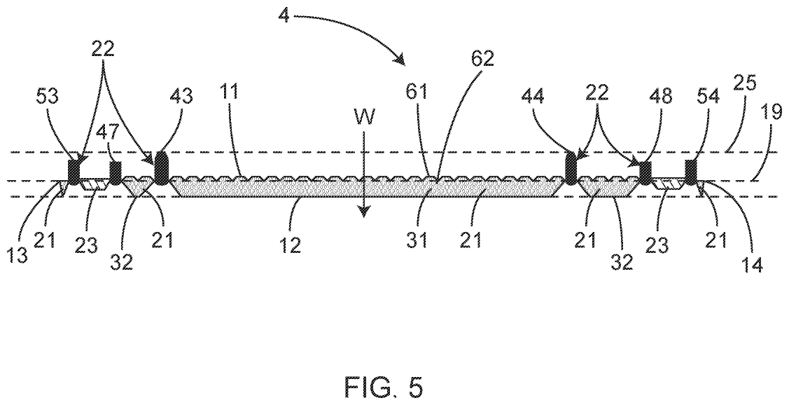

With reference to the example of FIGS. 2-5, in particular FIG. 5, the upper wicking layer 4 includes a top face 11 and an opposite bottom face 12 defining a through-thickness or wicking direction W of the upper wicking layer 4 therebetween. As can be seen for example in FIGS. 2 to 5, the upper wicking layer 4 includes a front edge 13, a rear edge 14 and two side edges 15 and 16 extending therebetween. A longitudinal direction L is a direction spanning between the front edge 13 and the rear edge 14, and a transverse direction T is a direction spanning between the two side edges 15 and 16. A longitudinal central axis 17 extends in the longitudinal direction L between the front edge 13 and the rear edge 14, so as to bisect the upper wicking layer 4 evenly. A transverse central axis 18 extends between the two side edges 15 and 16, crossing the longitudinal central axis 17 at a central intersection point P of the upper wicking layer 4. As shown in FIG. 5, a reference plane 19 is located between the top face 11 and the bottom face 12.

The upper wicking layer 4 can be fabricated using a knitting machine, such as a flat knitting machine, a linear knitting machine and/or a circular knitting machine. The circular knitting machine may comprise in a non-limiting example, a single jersey, weft knitting circular knitting machine, such as is available from Santoni® in a non-limiting example, and uses a technique to produce a seamless tubular fabric piece comprising a pattern of areas or zones knitted so as to have different liquid permeability in the through-thickness direction (W). The circular knitting machine performs the circular knitting process.

In some embodiments, the circular knitting machine may comprise in a non-limiting example, a double jersey, weft knitting circular knitting machine. In some embodiments, the circular knitting machine may comprise in a non-limiting example, a warp knitting machine.

The pattern of areas may be formed by selectively manipulating and arranging threads of the tubular fabric piece relative to each other during the circular knitting process. Additionally, the pattern can be further formed by subsequent processing of the tubular fabric piece, for example through one or more of dyeing, heating or applying a chemical treatment. The pattern can include zones, or knitted areas, of different degrees of permeability, e.g. of high transfer rate capability or high (relatively higher) permeability 21, 23 and zones of restricted (relatively lower) permeability 22, the different zones, or areas, being mutually arranged so as to inhibit movement of liquid towards the side edges 15, 16 at least along a portion of their length. In some embodiments the relatively high permeability area 21 comprises an intermediate permeability 23 area.

The permeability may comprise a measure of the liquid permeability through the layer 4, such that at the high transfer rate capability areas or high permeability areas 21, 23, the degree of liquid permeability is greater than the degree of liquid permeability in the restricted permeability area 22.

It is appreciated that the areas of high permeability 21 are arranged alongside the areas of restricted permeability 22 and/or areas of intermediate permeability 23 forming the upper layer 4 of varying topography, as seen in FIG. 5. It is noted that the varying topography may be formed by a knitted fabric comprised of a single layer, namely a single layer jersey without requiring a multi-layered knitted fabric. Forming the knotted fabric of the upper layer 4 with varying topography, yet of a single layer, is achievable by selectively manipulating and arranging threads of the tubular fabric piece relative to each other during the circular knitting process. As shown for example in FIG. 5, at least a majority of the areas of high permeability 21, 23 extend, or span, a depth parallel to the wicking direction W, from above the reference plane 19, in a first direction, e.g. downwardly, through the reference plane 19 towards the bottom face 12. At least a majority of the areas of restricted permeability 22 extend, or span, a depth parallel to the wicking direction W, from below the reference plane 19, upwardly through the reference plane 19 towards the top face 11. For the sake of clarity, the wicking direction W extends from the inside of the layer 4 or top face 11 in a first direction, e.g. downwardly towards the outside of the layer 4 or the bottom face 12.

The degree of permeability may be achieved in many different manners, e,g, by selecting a knitting density, topography and profile of the knitting, knitting patterns and/or number of layers/jerseys and the like. In one embodiment, the permeability is provided by a combination including knitting density and profile of the layer in a cross-section thereof taken perpendicular to the reference plane 19. The pattern of areas can comprise at least one area of relatively high permeability 21, knitted so as to facilitate passage of liquid in the through-thickness direction (W).

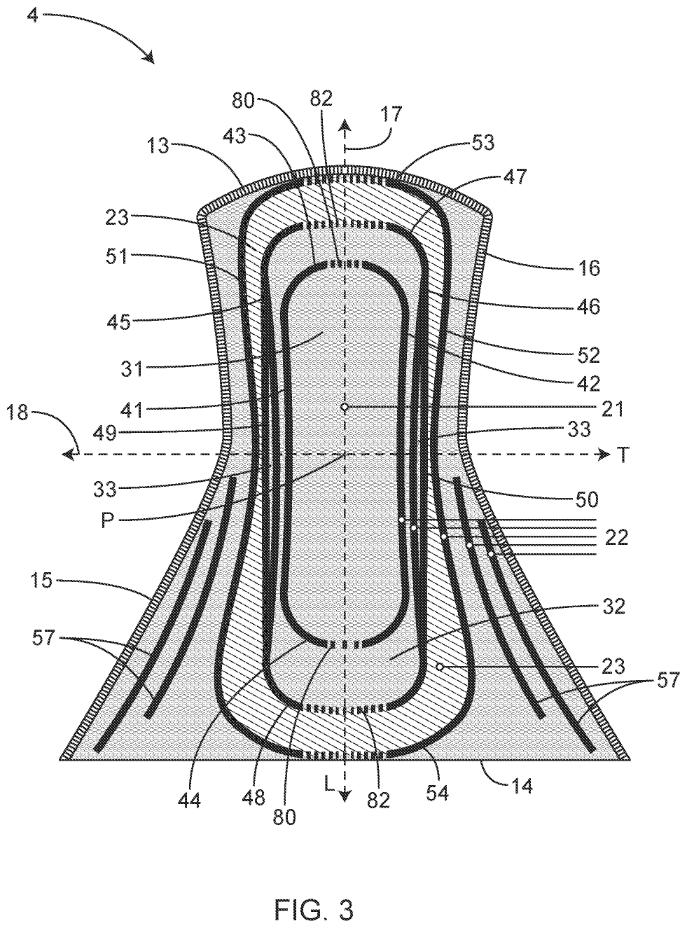

The at least one area of relatively high permeability 21 can include a first wicking zone 31 extending in the longitudinal direction L along and on either side of the longitudinal central axis 17, spaced from the side edges 15 and 16, and located centrally in the transverse direction T, as seen for example in FIGS. 3 and 5.

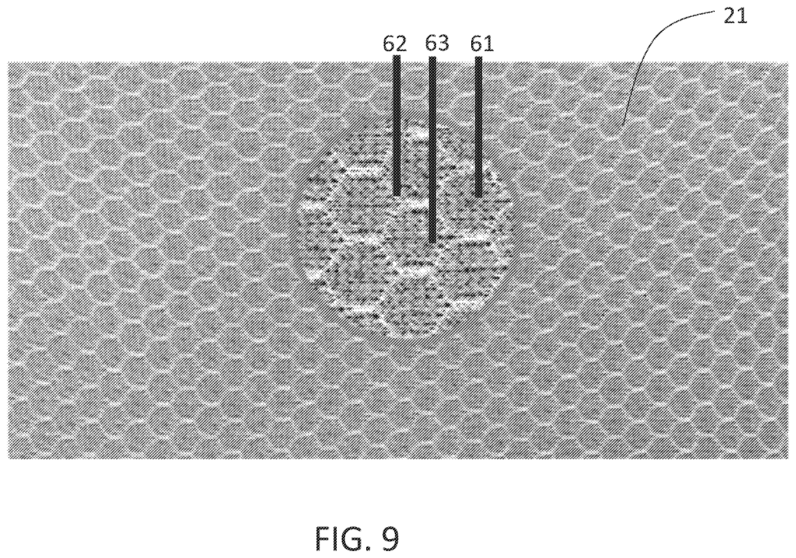

At least two areas of restricted, i.e. lesser, permeability 22 can include first and second raised zones or ridges 41 and 42 extending so as to bound the first wicking zone 31 at least partially along longitudinal side edges thereof, the at least two raised zones 41 and 42 being knitted in a different arrangement from that of the first wicking zone 31, so as to inhibit passage of liquid laterally from the first wicking zone 31 towards the side edges 15 and 16. The at least one area of relatively high permeability 21 can have a thickness extending from the reference plane 19 towards the bottom face 12, and comprises a textured surface having upward protrusions 61 towards the reference plane 19 and downward recesses 62 towards the bottom face 12. Optionally the recesses 62 can comprise recessed channels, and the recessed channels can circumscribe the protrusions 61, as shown in FIG. 9.

At least two areas of restricted permeability 22 can protrude outwardly from the reference plane 19, starting from below the reference plane 19, extending upwardly across the reference plane 19 to an upper plane 25 higher than the areas of high permeability 21 in the top face 11. The at least two areas of restricted permeability 22 can extend along at least a part of the sides of, and at least partially bounding, the at least one area of relatively high permeability 21, such as the first wicking zone 31 (shown in FIGS. 3 to 5). In a non-limiting example, the diameter of upward protrusion 61 can be a protrusion of 1-4 mm or in a non-limiting example 1-3 mm, and a width (measured in the transverse direction T) of the at least two areas of lower transfer rate capability or restricted permeability 22 can be 2 to 5 mm, for example.

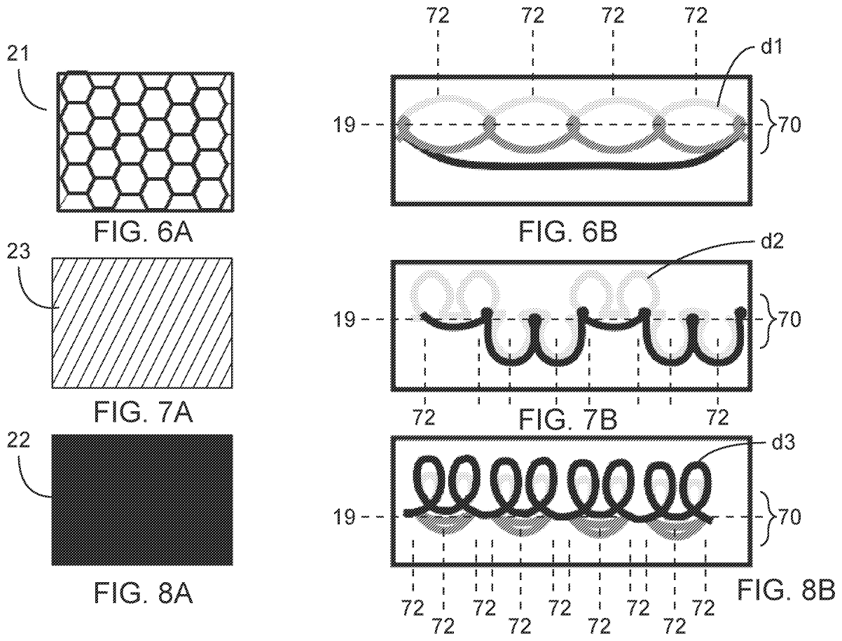

The different degrees of permeability may be defined in some embodiments by different degrees of porosity which for a predetermined yarn thickness is determined by a combination including knitting density and profile of the layer in a cross-section thereof taken perpendicular to the reference plane 19. The knitting density is determined by at least one or more of: the number of pores per unit area, namely the number of courses (horizontal rows) 70 (shown in exemplary area of high permeability in FIGS. 6A-8B) and the number of wales (vertical columns) 72 per unit area. The porosity may further define a thickness of a selected yarn forming the knitted fabric.

In a non-limiting example, the texture of the knitted fabric, namely pattern, is shown in FIGS. 6A, 7A and 8A by way of example, it being appreciated that the textures are not shown to scale and are not an exact representation. The first texture (FIG. 6A) of high permeability area 21 comprises a honeycomb texture, which may include different upper and lower yarns looping together on opposite sides of the reference plane 19 at the same number of wales/cm, and a third yarn defining a long floating yarn looping together below the reference plane 19 at every 3-5 loops of the upper and lower yarns. The second texture (FIG. 7A) of intermediate permeability area 23 comprises a twill texture (also referred to as a “twilled rib texture”) comprising a pattern of diagonal parallel ribs, which can include both ridges extending upwardly from the reference plane 19 comprising loops of a first yarn, and channels extending downwardly from the reference plane 19 comprising loops of the first yarn and loops of a second yarn. The second yarn can include a short floating yarn between pairs of the upwardly extending loops of the first yarn. The second texture (FIGS. 7A & B) has a larger wales/cm than the first texture (FIGS. 6A & B), e.g. 1.5× to 4×, preferably 2×. The third texture (FIG. 8A) of restricted permeability area 22 comprises a rib knit formed with alternating raised and lower rows (also referred to as “raised rib” or “raised line”). The third texture can include loops from two different yarns extending upwardly from the reference plane 19 with an even larger wales/cm than the first texture (FIGS. 6A & B) or the second texture (FIGS. 7A & B), e.g. 1.5× to 4×, preferably 2× the second texture, and 3× to 5×, preferably 4× the first texture. The third texture (FIGS. 8A & B) can also include loops from a third different yarn extending downwardly from the reference plane 19 with a shorter height and fewer loops forming a dense floating yarn.

It is appreciated that the texture of the knitted fabric, namely pattern, can be achieved in many by many different arrangements of yarns and FIGS. 6A-8B are shown by way of example.

In a non-limiting example, the cross-sectional profile is shown in FIGS. 6B, 7B and 8B. The first cross sectional area (FIG. 6B) of high permeability area 21 comprises a loop of a diameter d1 resulting in a first wales/cm value. The second cross sectional area (FIG. 7B) of intermediate permeability area 23 comprises a loop of a diameter d2 resulting in a second, larger, wales/cm value. The third cross sectional area (FIG. 8B) of restricted permeability area 22 comprises a loop of a diameter d3. It is seen that the diameter d1 is larger than the diameter d2, which is larger than the diameter d3, resulting in a third, largest, wales/cm value. Accordingly, the loop diameter d1 and hence the porosity of the high permeability area 21 is greater than the loop diameter d2 and hence the porosity of the intermediate permeability area 23, which is greater than the loop diameter d3 and hence the porosity of the restricted permeability area 22.

With reference to FIG. 9, the at least one area of relatively high permeability 21 can have a tessellated texture, optionally comprising a grid or honeycomb arrangement of the protrusions 61 and recesses 62. The at least one area of relatively high permeability 21 can comprise a plurality of openings 63 formed in the protrusions 61 other than regular spacing between the threads. The openings 63 pass through the upper wicking layer 4 in the through-thickness direction W and thus are configured for passage of liquid therethrough. The recesses 62 can comprise cover yarn, e.g. 40D-70D/20F-35F nylon, more preferably 50D/24F nylon, without dye, which can be processed for example by washing in high temperature water, such as in a non-limiting example at a temperature of above 90° C., preferably 98° C., to shrink to provide a valley around each protrusion 61. The protrusions 61 can be formed from a jersey of graphene nylon yarn, e.g. 80D-200D/40-60F, more preferably 150D/48F, and the cover yarn pleated together. The plurality of openings 63 can be a formed in a rib of graphene polymer nylon yarn and cover yarn pleated together.

In a non-limiting example, the knitted fabric can comprise 50D spandex jersey covered with the cover yarn, e.g. 50D/24F nylon, and the graphene nylon yarn, e.g. 150D/48F graphene nylon yarn pleated together.

Denier (abbreviated D), a unit of measure for the linear mass density of fibers, is the mass in grams per 9000 metres of the fiber. (1 g/9000 m or 0.11 mg/m). F is the number of filaments in the yarn.

It is appreciated that the texture of the relatively high permeability 21, as well as of the intermediate permeability area 23 and restricted permeability area 22, can be achieved by alternative embodiments including: alternative arrangements, alternative sized of protrusions 61, recesses 62 and openings 63 and/or alternative processing thereof.

With reference to the examples in FIGS. 3, 4 and 5, the areas of relatively high permeability 21, area of intermediate permeability 23 and the at least two areas of restricted permeability 22 may be mutually arranged to form a pattern of areas in any suitable manner. In some embodiments, the at least two areas of restricted permeability 22 can be joined together at longitudinal ends thereof by front and/or rear laterally extending joining portions to form a loop-shape (FIG. 3). In particular, a third laterally-extending raised zone 43 can extend in the transverse direction T across the longitudinal central axis 17 between the first and second longitudinally-extending raised zones 41 and 42 between the transverse central axis 18 and the front edge 13, and a fourth laterally-extending raised zone 44 can extend in the transverse direction T across the longitudinal central axis 17 between the first and second raised zones 41 and 42 between the transverse central axis 18 and the rear edge 14.

At least one of the front and rear joining portions, e.g. third and fourth laterally-extending raised zones 43 and 44, can be knitted in a similar manner to the at least two areas of restricted permeability 22, and at least a portion of which can extend in a discontinuous manner, e.g. alternating between areas of restricted permeability and intermediate or higher permeability, so as to provide a port 80, e.g. an area of low to intermediate permeability, configured to restrictively allow passage of a liquid through the upper layer 4 at least in a longitudinal direction from one side of the respective joining portion to the other side.

In the example shown in FIG. 3, additional areas of restricted permeability 22 are provided as follows. Fifth and sixth longitudinally-extending raised zones or ridges 45 and 46 extend substantially parallel to the first and second longitudinally-extending raised zones 41 and 42, but are longer in length. Seventh and eighth laterally-extending raised zones 47 and 48, extending between the fifth and sixth longitudinally-extending raised zones 45 and 46 and substantially parallel to the third and fourth laterally-extending raised zones 43 and 44 are provided in a similar manner to the at least two areas of restricted permeability 22, and at least a portion of which can extend in a discontinuous manner, e.g. alternating between areas of restricted permeability and intermediate or higher permeability, so as to provide a port 82, e.g. areas of low to intermediate permeability, configured to restrictively allow passage of a liquid through the layer at least in a longitudinal direction from one side of the respective joining portion to the other side. The additional areas of restricted permeability 22 form an alternating arrangement of areas of relatively high permeability 21 with areas of restricted permeability 22 in both the lateral and longitudinal directions. Accordingly, a second wicking zone 32 can be provided on either side of and/or surrounding the first wicking zone 31, at least partially defined or bordered by the fifth and sixth longitudinally-extending raised zones 45 and 46 and the seventh and eighth laterally-extending raised zones 47 and 48.

A third wicking zone 33 can be provided on either side of and/or surrounding the first wicking zone 31 and the second wicking zone 32, e.g. in a central transverse area, defined by a ninth and tenth longitudinally-extending raised zones 49 and 50.

The pattern of areas can also include at least one area of intermediate permeability 23, which is knitted in a different arrangement from the at least one area of relatively high permeability 21 and the at least two areas of restricted permeability 22 so as to allow lateral movement of a liquid in a direction having a component in the longitudinal direction, and has a thickness extending from the reference plane 19 towards the bottom face 12 to a lesser extent than the at least one area of relatively high permeability 21, the at least one area of intermediate permeability 23 being at least partially bounded by at least one area of restricted permeability 22. The at least one area of intermediate permeability 23 can comprise a textured surface having alternately arranged ridges and channels, the ridges protruding upwardly towards the reference plane 19 and the channels being recessed downwardly towards the bottom face 12. Optionally, the ridges and channels are arranged extending at an angle, e.g. in the range of 30° to 60°, optionally at 45° between the longitudinal and transverse directions. The ridges and channels encourage lateral movement of fluid along the surface of the top face 11 away from the front edge 13 and rear edge 15 and towards the areas of higher permeability 22 and/or the ports 80 or 82 in both longitudinal and transverse directions.

Accordingly, the area of intermediate permeability 23 can be provided on either side of and/or surrounding the first wicking zone 31 and/or the second wicking zone 32 defined by an eleventh and twelfth longitudinally-extending raised zones or ridges 51 and 52 and a thirteenth and fourteenth laterally-extending raised zones or ridges 53 and 54.

Additional raised zones or ridges 57 can be provided between the first wicking zone 31 and/or the second wicking zone 32 and the side edges 15 and 16, which extend longitudinally or at an acute angle to the longitudinal direction L to further obstruct the flow of fluids from the central zones, e.g. 23, 31, 32 and/or 33, towards the side edges 15 and 16 of the upper wicking layer 4.

The upper wicking layer 4 can be comprised of threads made of any suitable component. In an exemplary embodiment the threads comprise at least one of graphene, nylon or spandex fibres. Graphene has antimicrobial properties and additionally has high moisture wicking capabilities. Graphene has a high carbon content and may comprise a Graphene polymer.

The upper wicking layer 4 can be coated with a chemically applied wicking finish.

A method of producing the upper wicking layer 4 for the reusable liquid-absorbent pad can comprise knitting a tubular fabric piece comprising the pattern of areas surrounded by cut guidelines along which the layer can be cut from the tubular fabric piece, the cut guidelines demarcating the front edge 13, the rear edge 14 and the two side edges 15 and 16 of the upper wicking layer 4, the knitting may comprise a circular weft knitting process, and subsequently cutting out the layer from the tubular fabric piece along the cut guidelines.

In a circular knitting process, a circular knitting pattern matching the design is created in a programming software. Each style and size can have its own program pattern created. This software pattern can be programmed to a disk or other form of non-transitory memory for insertion into a computerized system of a circular knitting machine for execution by a controller processor for performing the circular knitting process. The technician can set up the circular knitting machine with suitable yarn spools, and optionally performing a few inspections and test rounds, the machine knits the upper wicking layer 4 on a tubular seamless fabric piece. The tubular fabric piece is formed with cut guidelines along which the layer can be cut from the tubular fabric piece. The cut guidelines demarcate the front edge, the rear edge and the two side edges of the layer.

The tube is sent to a cutter to follow the cut guidelines of the upper wicking layer 4, and then the upper wicking layer 4 is applied to the additional layers of fabrics that comprise the at least one middle absorbent layer 5, and the lower liquid-impervious layer 6, to form an absorbent pad 3. In some examples, the layers of the absorbent pad, once stacked, are joined at their periphery prior to attachment to a panty gusset, such as by sewing, bonding by gluing, heat-pressing or the like. The attachment may be made in any suitable manner and the order of the attachment steps may be interchanged.

The absorbent pad 3 is then sewn into the gusset of the underwear or panty 2. Optionally, only some of the peripheral edges of the absorbent pad are fixed to the gusset of the panty, for example, only two or three-sides of the absorbent pad 3 can be stitched to the gusset of the underwear 2 to maintain flexibility and stretch of the underwear 2 relative to the rather less flexible pad 3, to enable a better conforming fit to a user wearing the pant. For example, two side edges of the pad and optionally a rear edge of the pad can be affixed to the underwear gusset, with at least a front edge of the pad remaining free and unattached to the underwear.

In some embodiments in a non-limiting example, the attachment of the pad to the underwear can for example be using a waterproof textured thread, which can help prevent leakage out of the sides of the pad.

It is appreciated that the visual appearance of the features of the reusable liquid-absorbent article shown in any one of FIGS. 1-9 can assume many presentations and alternatives and are not dictated solely by their technical function, rather by ornamental consideration as well. For example, the areas of permeability can be formed in a different shapes, textures, dimensions, colors, lengths and arrangements.

1.-22. (canceled)

23. A layer for a reusable liquid-absorbent article configured to constitute an upper layer of a pad intended for contact with a body of a user, the layer being formed of a knitted fabric, and comprising:
a top face and an opposite bottom face defining a through-thickness direction of the layer therebetween;
a front edge, a rear edge and two side edges extending therebetween, a longitudinal direction being a direction spanning between the front edge and the rear edge, and a transverse direction being a direction spanning between the two side edges;
a longitudinal central axis extending in the longitudinal direction between the front and rear edges so as to bisect the layer evenly;
a transverse central axis extending between the two side edges and crossing the longitudinal central axis at a central intersection point of the layer;
a pattern of knitted areas configured to have different degrees of liquid permeability in the through-thickness direction of the layer, including areas of high permeability and areas of restricted permeability, mutually arranged so as to inhibit movement of liquid towards the side edges at least along a portion of their length, each area having a height along the through-thickness direction; and
a reference plane passing between the top and bottom faces and parallel to the longitudinal central axis and the transverse central axis, at least a majority of the areas of high permeability being disposed with most of their height extending downwardly from the reference plane, and at least a majority of the areas of restricted permeability being disposed with most of their height extending upwardly from the reference plane,
wherein the layer is cut from a tubular fabric piece formed by a knitting process, and the pattern of knitted areas is formed by selectively manipulating and arranging threads of the tubular fabric piece relative to each other during the knitting process.

24. The layer according to claim 23, wherein said different degrees of liquid permeability is defined by different degrees of porosity, which for a predetermined yarn thickness is determined by a combination including: knitting density and profile of the layer in a cross-section thereof taken perpendicular to the reference plane.

25. The layer according to claim 23, wherein the reusable liquid-absorbent article comprises a crotch pad.

26. The layer according to claim 23, wherein the pattern of knitted areas comprises:
at least one area of relatively high permeability, configured to facilitate passage of liquid in the through-thickness direction of the layer, the at least one area of relatively high permeability extending in the longitudinal direction and being spaced from the two side edges and located centrally in the transverse direction; and
at least two areas of restricted permeability extending so as to bound the at least one area of relatively high permeability at least partially along longitudinal sides thereof, the at least two areas of restricted permeability being knitted in a different arrangement from that of the at least one area of relatively high permeability, so as to inhibit passage of a liquid laterally from the at least one area of relatively high permeability towards the two side edges.

27. The layer according to claim 26, wherein the at least one area of relatively high permeability has a height extending from above the reference plane downwardly below the reference plane, and comprises a first textured surface having protrusions extending above the reference plane, and recesses extending downwardly below the reference plane, optionally wherein the recesses comprise recessed channels, further optionally wherein the recessed channels circumscribe the protrusions.

28. The layer according to claim 27, wherein the at least one area of relatively high permeability has a tessellated texture, optionally comprising a grid or honeycomb arrangement of the protrusions and the recesses.

29. The layer according to claim 27, wherein the at least one area of relatively high permeability comprises a plurality of openings formed in the protrusions other than regular spacing between the threads, the openings passing through the layer in the through-thickness direction, configured for passage of liquid therethrough.

30. The layer according to claim 26, wherein the at least two areas of restricted permeability have a height extending from below the reference plane towards the top face.

31. The layer according to claim 26, wherein the at least two areas of restricted permeability are joined together at longitudinal ends thereof by front and rear joining portions to form a loop-shape.

32. The layer according to claim 31 wherein at least one of the front and rear joining portions of the loop-shape is knitted in a similar manner to the at least two areas of restricted permeability and extends in a discontinuous manner, so as to provide an area of low permeability, configured to restrictively allow passage of liquid through the layer at least in the longitudinal direction from one side of the respective joining portion to another side.

33. The layer according to claim 26, further comprising at least one area of intermediate permeability which is knitted in a different arrangement from the at least one area of relatively high permeability and the at least two areas of restricted permeability, so as to allow lateral movement of liquid in a direction having a component in the longitudinal direction, and has a height extending from above the reference plane towards the bottom face to a lesser extent than the at least one area of relatively high permeability, the at least one area of intermediate permeability being at least partially bounded by at least one area of restricted permeability.

33. The layer according to claim 33, wherein the at least one area of intermediate permeability comprises a second textured surface having alternately arranged ridges and channels, the ridges protruding upwardly above the reference plane and the channels being recessed downwardly towards the bottom face, optionally wherein the ridges and channels are arranged extending at an angle between the longitudinal and transverse directions.

35. The layer according to claim 33, wherein the at least one area of intermediate permeability has a twilled rib texture.

36. The layer according to claim 23, wherein the area of restricted permeability has a raised line texture.

37. The layer according to claim 23, wherein the threads comprise at least one of graphene, nylon or spandex fibres.

38. The layer according to claim 23, comprising a chemically applied wicking finish.

39. The layer according to claim 23, wherein the knitting process is a circular knitting process.

40. A method of producing a layer for a reusable liquid-absorbent article according to claim 23, comprising:
knitting a tubular fabric piece comprising the pattern of areas surrounded by cut guidelines along which the layer can be cut from the tubular fabric piece, the cut guidelines demarcating the front edge, the rear edge and the two side edges of the layer;
wherein knitting comprises a circular weft knitting process; and
subsequently cutting out the layer from the tubular fabric piece along the cut guidelines.

41. An upper wicking layer for reusable absorbent underwear configured for contact with a body of a user, the upper wicking layer being cut from a circular knitted fabric and comprising:
side edges, a front and a rear,
a first zone of higher permeability at least partially bounded by a second zone of lesser permeability on one side thereof and a third zone of lesser permeability on another side thereof, the second zone of lesser permeability and the third zone of lesser permeability extending outwardly from either side of the first zone configured to inhibit movement of liquid applied to the first zone towards the side edges;
wherein the first zone of higher permeability has:
a first degree of porosity of the knitted fabric;
wherein the second and third zones of lesser permeability have:
a second degree of porosity of the knitted fabric, the first degree of porosity is greater than the second degree of porosity.
